(19) United States
(12) Patent Application Publication
(10) Pub. No.: US 2014/0171708 A1
(43) Pub. Date: Jun. 19, 2014

(54) SYSTEM AND PROCESS FOR CONVERTING NATURAL GAS INTO BENZENE

(71) Applicant: Ceramatec, Inc., Salt Lake City, UT (US)

(72) Inventors: Pallavi Chitta, West Valley City, UT (US); Mukund Karanjkar, West Valley City, UT (US)

(73) Assignee: Ceramatec, Inc., Salt Lake City, UT (US)

(21) Appl. No.: 14/090,776

(22) Filed: Nov. 26, 2013

Related U.S. Application Data
(60) Provisional application No. 61/731,397, filed on Nov. 29, 2012.

(51) Int. Cl. C07C 2/76

(52) U.S. Cl. CPC C07C 2/76
USPC 585/417; 422/187

(57) ABSTRACT

A system and process to produce an aromatic hydrocarbon via catalyzed nonoxidative dehydroaromatization (DHA). The system includes a reaction zone containing a dehydroaromatization catalyst. A reactant feed stream inlet supplies a reactant composition, such as natural gas, to the reaction zone. A heater maintains the reaction zone at a suitable dehydroaromatization temperature. A product stream exit removes the aromatic hydrocarbon produced by the nonoxidative dehydroaromatization of the reactant composition from the reaction zone. A hydrogen separation membrane is disposed between the reaction zone and a hydrogen stream exit to enable continuous and selective removal of hydrogen produced in the reaction zone. A hydrogen recycle stream diverts a portion of hydrogen from the hydrogen stream exit and adds the portion of hydrogen to the reactant composition supplied to the reaction zone. The hydrogen may also be used to regenerate the dehydroaromatization catalyst.

### CROSS-REFERENCE TO RELATED APPLICATIONS

This application claims the benefit of U.S. Provisional Patent Application No. 61/731,397, filed Nov. 29, 2012, entitled NATURAL GAS TO BENZENE. The foregoing application is incorporated by reference.

### FIELD OF THE INVENTION

The present invention relates to producing benzene from natural gas or methane. More particularly, the present invention produces benzene via dehydroaromatization (DHA) of methane in high yields with continuous hydrogen removal.

### BACKGROUND OF THE INVENTION

Currently, petroleum crude costs six to eight times more than natural gas on an energy content basis. Moreover, approximately 97% of natural gas is currently produced from domestic sources, whereas more than 50% of the crude oil demand is imported. This presents opportunities for reduction in petroleum crude usage and has led to the emergence of new processes with more attractive economics for producing value-added chemicals and fuels from natural gas.

Benzene, which is currently produced from crude oil, is a chemical of great industrial importance with current global consumption in excess of 30 million metric tons per annum and net growth of 4% annually, leading to a total market size of more than $50,000,000,000. It is a starting material for nylons, polycarbonates, polystyrene and epoxy resins. Also, benzene can be directly converted to aniline, chlorobenzene, maleic anhydride, succinic acid, and countless other useful industrial chemicals. Benzene is a gasoline component and can be converted to cyclohexane, another gasoline component via a commercial process.

Benzene can be synthesized from natural gas (methane) using a catalyst in a single step via dehydroaromatization (DHA) route in the absence of oxygen as follows.

6CH₄→C₆H₆+9H₂

While the DHA process is commercially very attractive, there are two primary technical commercialization challenges for this reaction:

Kinetic: As hydrogen is removed, a coking reaction on the catalyst surface competes with the desired DHA reaction; and

Thermodynamic: Equilibrium conversion of methane to benzene is limited to about 12% at 700° C. and 1 atmosphere.

There is a need in the art for further advances in the DHA process which overcome the kinetic and thermodynamic challenges and which improve the yield of benzene and which limit coking of the catalyst.

### BRIEF SUMMARY OF THE INVENTION

The disclosed invention relates to a system and process to produce an aromatic hydrocarbon via catalyzed nonoxidative dehydroaromatization (DHA). The system includes a reaction zone containing a dehydroaromatization catalyst. A reactant feed stream inlet supplies a reactant composition to the reaction zone. A heater maintains the reaction zone at a suitable dehydroaromatization temperature. A product stream exit removes the aromatic hydrocarbon produced by the nonoxidative dehydroaromatization of the reactant composition from the reaction zone. A hydrogen separation membrane is disposed between the reaction zone and a hydrogen stream exit. The hydrogen separation membrane selectively removes hydrogen produced in the reaction zone. A hydrogen recycle stream diverts a portion of hydrogen from the hydrogen stream exit and adds the portion of hydrogen to the reactant composition supplied to the reaction zone. The hydrogen may also be used to regenerate the dehydroaromatization catalyst.

Dehydroaromatization catalysts are known. Some suitable catalysts are metal/zeolite catalysts based on HZSM-5 zeolites. Several different metals have been proposed, including molybdenum, tungsten, rhenium, vanadium, and zinc, with the HZSM-5 zeolites. The rhenium exchanged zeolite (Re/ZSM-5) catalyst is a presently preferred dehydroaromatization catalyst.

The hydrogen separation membrane is a ceramic membrane that selectively transports H⁺ ions at dehydroaromatization operating temperatures. In some non-limiting embodiments, the hydrogen separation membrane is thermally stable and effective at a temperature above 800° C. In some non-limiting embodiments, the hydrogen separation membrane selectively transports H⁺ ions under a hydrogen partial pressure gradient, a concentration gradient, or an applied voltage. Non-limiting examples of materials from which the hydrogen separation membrane is fabricated include a perovskite, a doped cerate, a doped zirconate, or an acidic phosphate. In one non-limiting embodiment, the hydrogen separation membrane comprises a barium-cerate ceramic composite. The membrane may comprise a 10-30 μm pinhole-free dense membrane.

The reactant may comprise one or more C₁-C₄ alkanes, including by not limited to methane, ethane, propane, and butane. In one non-limiting embodiment, the reactant comprises natural gas.

The dehydroaromatization reaction typically occurs at a temperature in the range from about 500° C. to 1000° C. In some embodiments, the dehydroaromatization reaction occurs at a temperature in the range from about 700° C. to 900° C.

The disclosed catalyzed nonoxidative dehydroaromatization aromatic hydrocarbon typically produces one or more aromatic hydrocarbons. The produced hydrocarbon may include benzene, toluene, ethylbenzene, styrene, xylene or naphthalene. The disclosed reaction may also result in a benzene precursor, such as ethylene.

The kinetic challenge of the dehydroaromatization reaction is solved by using a highly active and benzene selective coke resistant catalyst. The equilibrium limitation of the dehydroaromatization reaction is overcome through the continuous selective separation of hydrogen from the reaction zone at reaction temperatures. As hydrogen is continuously removed, up to 100% single-pass conversion becomes possible from the thermodynamic vantage point. The dehydroaromatization catalyst and hydrogen separation synergy dramatically improves the commercialization potential of this disclosed process.

The disclosed system may be used in a process for catalyzed nonoxidative dehydroaromatization (DHA) of a reactant feed stream. As noted above, the reactant feed stream may comprise one or more C₁-C₄ alkanes, including but not limited to methane, ethane, propane, and butane. In some instances the reactant feed stream comprises natural gas.

In the disclosed process the reactant feed stream is brought in contact with a dehydroaromatization catalyst in a reaction zone under conditions to produce an aromatic hydrocarbon and hydrogen. Hydrogen is continuously removed from the reaction zone through a hydrogen separation membrane and collected. A reduced pressure or vacuum may be applied to facilitate hydrogen removal and collection. The produced aromatic hydrocarbon as described above is continuously removed from the reaction zone in a product stream.

The disclosed process may include adding a portion of the separated hydrogen to the reactant feed stream to help control formation of the desired aromatic hydrocarbon. The process further includes heating the reaction zone to a suitable dehydroaromatization temperature. The disclosed process may further include periodically regenerating the dehydroaromatization catalyst by contacting the dehydroaromatization catalyst with hydrogen.

These features and advantages of the present invention will become more fully apparent from the following description and appended claims, or may be learned by the practice of the invention as set forth hereinafter.

### BRIEF DESCRIPTION OF THE SEVERAL DRAWINGS

In order that the manner in which the above-recited and other features and advantages of the invention are obtained and will be readily understood, a more particular description of the invention briefly described above will be rendered by reference to specific embodiments thereof that are illustrated in the appended drawings. Understanding that the drawings are not made to scale, depict only some representative embodiments of the invention, and are not therefore to be considered to be limiting of its scope, the invention will be described and explained with additional specificity and detail through the use of the accompanying drawings in which:

FIG. 1 depicts the combination of a hydrogen separation membrane and a dehydroaromatization catalyst usable in the disclosed system and process.

FIG. 2 depicts a system for efficient dehydroaromatization of a reactant composition to produce an aromatic hydrocarbon using a combination of a hydrogen separation membrane and a dehydroaromatization catalyst.

FIG. 3 depicts another system for efficient dehydroaromatization of a reactant composition to produce an aromatic hydrocarbon using a combination of a hydrogen separation membrane and a dehydroaromatization catalyst.

FIG. 4 is a schematic representation of a system for dehydroaromatization of a reactant composition to produce an aromatic hydrocarbon.

FIG. 5 is schematic representation of a CHEMKIN® chemical model and simulation methodology for the disclosed system and process.

FIG. 6 is a graph of experimental results compared with chemical model simulated product selectivities as a function of methane conversion.

FIG. 7 is a conceptual model of six equilibrium reactors used by chemical process simulation software to evaluate methane conversion as a function of percentage hydrogen removal.

FIG. 8 is a graph showing the results of the chemical process simulation evaluate methane conversion as a function of percentage hydrogen removal.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment. Additionally, while the following description refers to several embodiments and examples of the various components and aspects of the described invention, all of the described embodiments and examples are to be considered, in all respects, as illustrative only and not as being limiting in any manner.

Furthermore, the described features, structures, or characteristics of the invention may be combined in any suitable manner in one or more embodiments. In the following description, numerous specific details are provided, such as examples of suitable dehydroaromatization catalysts, hydrogen separation membrane materials, operating conditions and variations, etc., to provide a thorough understanding of embodiments of the invention. One having ordinary skill in the relevant art will recognize, however, that the invention may be practiced without one or more of the specific details, or with other processes, components, materials, and so forth. In other instances, well-known structures, materials, or operations are not shown or described in detail to avoid obscuring aspects of the invention.

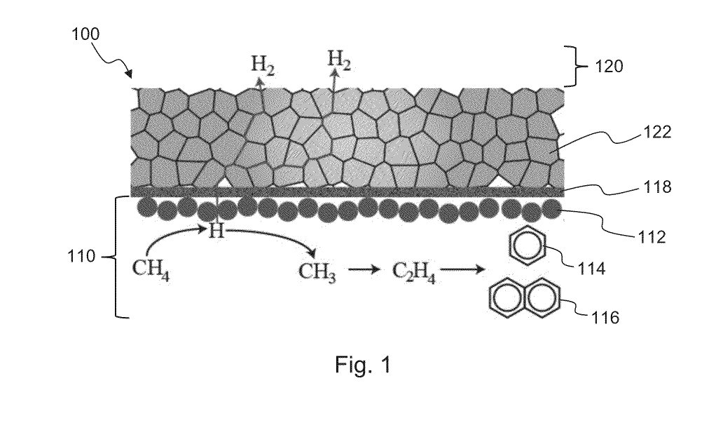

A system and process to produce an aromatic hydrocarbon via catalyzed nonoxidative dehydroaromatization (DHA) are disclosed herein. FIG. 1 depicts some features of the system and process schematically. The system 100 includes a reaction zone 110 containing a dehydroaromatization catalyst 112. A reactant composition is supplied to the reaction zone 110. The reactant composition may comprise one or more C₁-C₄ alkanes, including by not limited to methane, ethane, propane, and butane. In one non-limiting embodiment, the reactant comprises natural gas. To simplify the disclosure, FIG. 1 depicts a reactant composition comprising methane. Methane undergoes dehydroaromatization in the presence of catalyst 112 to produce one or more aromatic hydrocarbons. Intermediate compounds are typically produced, such as methane radical (.CH₃) and ethylene (C₂H₄). FIG. 1 depicts the formation of the aromatic hydrocarbons benzene 114 and naphthalene 116.

The dehydroaromatization reaction releases hydrogen. A hydrogen separation membrane 118 is disposed between the reaction zone 110 and a hydrogen stream exit 120. Thus the reaction zone 110 is on the retentate side of the membrane 118 and the hydrogen stream exit 120 is on the permeate side of the membrane. The hydrogen separation membrane 118 selectively removes hydrogen produced in the reaction zone 110. FIG. 1 depicts the hydrogen separation membrane 118 disposed on a porous substrate 122. A porous substrate may be advantageous depending upon the strength and thickness of the membrane 118.

Dehydroaromatization catalysts 112 are known. Some suitable catalysts are metal/zeolite catalysts based on HZSM-5 zeolites. Several different metals have been proposed, including molybdenum, tungsten, rhenium, vanadium, and zinc, with the HZSM-5 zeolites. The rhenium exchanged zeolite (Re/ZSM-5) catalyst is a presently preferred dehydroaromatization catalyst because Re-based H-ZSM5 systems are superior in reactivity, selectivity and stability than the Mo-based systems.

The hydrogen separation membrane 118 is a ceramic membrane that selectively transports H⁺ ions at dehydroaromatization operating temperatures. A variety of metallic, ceramic and polymer membranes have been used for H₂ separation from gas streams. The most common metallic membrane materials are palladium (Pd) and palladium alloys. However, these materials are expensive, strategic and less suitable for H₂ separation from dehydroaromatization reaction since Pd promotes coking. A number of organic membranes (e.g. Nafion® a registered mark of the Dupont Corporation) have also been identified as protonic conductors, but these are limited to lower temperature applications (less than 150° C.). The invention preferably uses a ceramic hydrogen separation membrane 118 that can withstand operation temperatures under a wide range of high-temperatures and that are suitable for promoting the DHA reaction. In some non-limiting embodiments, the hydrogen separation membrane 118 is thermally stable and effective at a temperature above 800° C. In some non-limiting embodiments, the hydrogen separation membrane 118 selectively transports H⁺ ions under a hydrogen partial pressure gradient, a concentration gradient, or an applied voltage. Non-limiting examples of materials from which the hydrogen separation membrane 118 is fabricated include a perovskite, a doped cerate, a doped zirconate, or an acidic phosphate. In one non-limiting embodiment, the hydrogen separation membrane 118 comprises barium. In another non-limiting embodiment, the membrane 118 comprises cerate. In another non-limiting embodiment, the membrane 118 is a composite comprising BaCeO₃ and an electronic conducting phase. The membrane may comprise a 10-30 μm pinhole-free dense membrane.

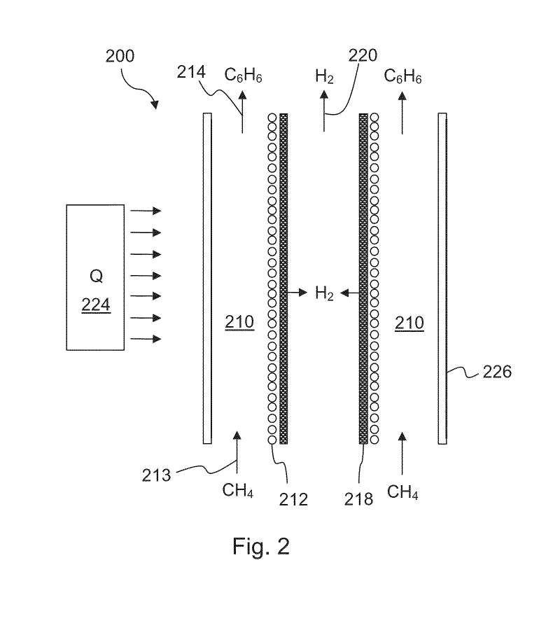

FIG. 2 illustrates one non-limiting system to produce an aromatic hydrocarbon via catalyzed nonoxidative dehydroaromatization. The system 200 includes a reaction zone 210 containing a dehydroaromatization catalyst 212. A reactant composition 213 is supplied to the reaction zone 210. The reactant composition 213 may comprise one or more C₁-C₄ alkanes, including by not limited to methane, ethane, propane, and butane. In one non-limiting embodiment, the reactant composition comprises natural gas. FIG. 2 depicts a reactant composition 213 comprising methane. Methane undergoes dehydroaromatization in the presence of catalyst 212 to produce one or more aromatic hydrocarbons 214. For simplicity, FIG. 2 depicts the formation of benzene (C₆H₆) as the aromatic hydrocarbon 214. Other aromatic hydrocarbons may be produced, including but not limited to, toluene, ethylbenzene, styrene, xylene or naphthalene. The dehydroaromatization reaction may also produce a benzene precursor, such as ethylene.

The dehydroaromatization reaction of methane releases hydrogen according to the reaction, 6CH₄→C₆H₆+9H₂. A hydrogen separation membrane 218 is disposed between the reaction zone 210 and a hydrogen stream exit 220. A vacuum or negative pressure may be applied to the hydrogen stream exit 220 to facilitate hydrogen removal. The pressure differential may also facilitate hydrogen transporting across the hydrogen separation membrane 218 from the reaction zone 210 to the hydrogen stream exit 220.

A heater 224 may be provided to control and maintain the reaction zone 210 at a suitable dehydroaromatization temperature. The dehydroaromatization reaction typically occurs at a temperature in the range from about 500° C. to 1000° C. In some embodiments, the dehydroaromatization reaction occurs at a temperature in the range from about 700° C. to 900° C.

The system 200 depicted in FIG. 2 shows a center hydrogen stream exit 220 surrounded by the reaction zone 210. This configuration may be constructed using concentric tubes, with the center tube being fabricated of a ceramic hydrogen separation membrane material and the outer tube being fabricated of a suitable temperature resistant and inert material. Alternatively, the configuration shown in FIG. 2 may be constructed of parallel plates with suitable sidewalls and seals to form the disclosed reaction zone 210 and hydrogen stream exit 220. While, FIG. 2 depicts the dehydroaromatization catalyst 212 disposed in close proximity to the hydrogen separation membrane 218, it is to be understood that the relative sizes and distances shown in FIG. 2 are for illustration only. In some non-limiting embodiments, the catalyst may substantially fill the reaction zone 210. In other non-limiting embodiments, the outer walls 226 may be disposed close to the catalyst.

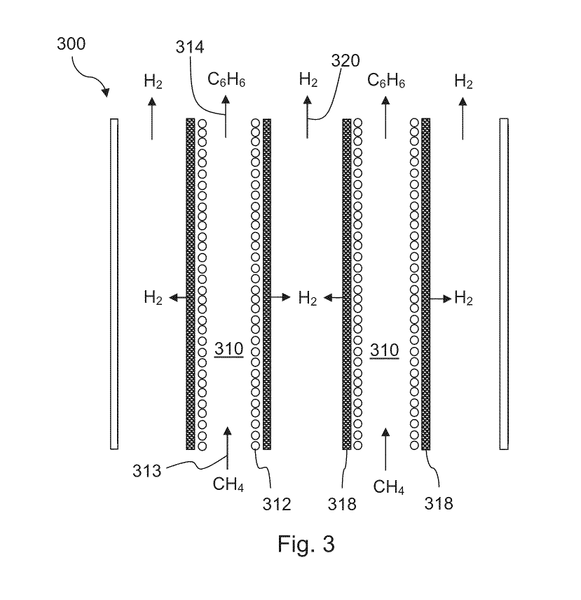

Many alternative configurations may be utilized which combine a dehydroaromatization catalyst and hydrogen separation membrane. FIG. 3 depicts another non-limiting system to produce an aromatic hydrocarbon via catalyzed nonoxidative dehydroaromatization. The system 300 of FIG. 3 is a variation of the system 200 of FIG. 2 and not all common features are illustrated and discussed below. The system 300 includes a reaction zone 310 containing a dehydroaromatization catalyst 312. A reactant composition 313 is supplied to the reaction zone 310. FIG. 3 depicts a reactant composition 313 comprising methane. Methane undergoes dehydroaromatization in the presence of catalyst 312 to produce one or more aromatic hydrocarbons 314. The dehydroaromatization reaction of methane releases hydrogen. A hydrogen separation membrane 318 is disposed between the reaction zone 310 and a hydrogen stream exit 320. As mentioned above, the reaction zone 310 is on the retentate side of the membrane 318 and the hydrogen stream exit 320 is on the permeate side of the membrane.

The system 300 depicted in FIG. 3 shows multiple reaction zones 310, multiple hydrogen separation membranes 318, and multiple hydrogen stream exits 320. It will be appreciated that even more reaction zones 310, combined with hydrogen separation membranes 318 and hydrogen stream exits 320, may be included in alternative systems. Such configurations may be constructed of stacked parallel plates with suitable sidewalls and seals to form the disclosed reaction zones 310 and hydrogen stream exits 320.

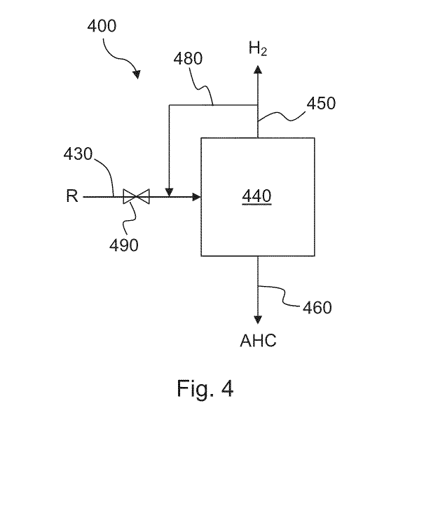

FIG. 4 shows a schematic representation of a non-limiting system 400 to produce an aromatic hydrocarbon (AHC) via catalyzed nonoxidative dehydroaromatization. The systems disclosed in FIGS. 1-3 discussed above, as well as modifications and variations within the level of skill in the art, may be utilized in the system 400 shown in FIG. 4. A reactant feed stream 430 supplies a reactant composition (R) to a reactor 440. The reactor 440 includes one or more reaction zones dehydroaromatization catalyst as disclosed above. The reactor 440 also includes one or more hydrogen separation membranes which enable continuous removal of hydrogen from the reaction zone(s). A hydrogen stream exit 450, which may provide collection of hydrogen from multiple reaction zones, allows for removal and recovery of hydrogen produced during the dehydroaromatization reaction. A product stream exit 460 removes the aromatic hydrocarbon (AHC) produced by the nonoxidative dehydroaromatization of the reactant composition (R) from the reactor 440. A hydrogen recycle stream 480 diverts a portion of hydrogen from the hydrogen stream exit 450 and adds the portion of hydrogen to the reactant composition (R) supplied to the reactor 440.

The hydrogen may also be used to regenerate the dehydroaromatization catalyst. As hydrogen is removed from reactant composition, the resulting hydrocarbon becomes more carbon-rich until coke is formed on the catalyst. Coke deactivates the catalyst. The catalyst may be regenerated by exposing the coke with hydrogen and forming methane according to the following reaction: C+2H₂→CH₄. Catalyst regeneration may be achieved by closing the supply of reactant composition to the reactor with valve 490 and instead supplying hydrogen via the recycle stream 480. To enable continuous operation multiple systems 400 may be used in parallel or series such that while one system is stopped to regenerate the catalyst, other systems may continue operation uninterrupted.

The following examples are given to illustrate various embodiments within, and aspects of, the scope of the present invention. These are given by way of example only, and it is understood that the following examples are not comprehensive or exhaustive of the many types of embodiments of the present invention that can be prepared in accordance with the present invention.

### EXAMPLE 1

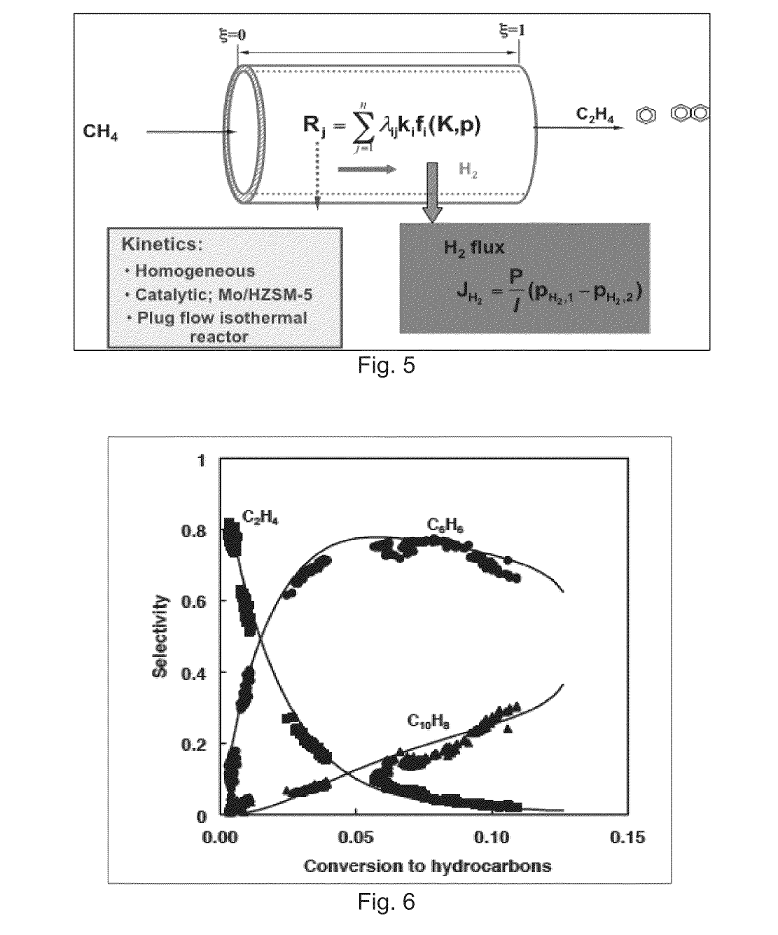

Computer chemical reaction simulation studies were performed to model and examine the operation of the dehydroaromatization reaction using the combination of a dehydroaromatization catalyst with a hydrogen separation membrane for continuous hydrogen removal from the reaction zone. Chemical reaction simulation software, CHEMKIN®, provided by Reaction Design, San Diego, Calif. was used to perform the chemical reaction simulation studies. The CHEMKIN® model and simulations methodology is shown as a schematic in FIG. 5.

The CHEMKIN® chemistry simulation results suggest that bi-functional catalysts, such as Metal/H-ZSM5, with continuous H₂ removal provide almost complete CH₄ conversion at practical residence time (100 s) and intermediate values of dimensionless transport rates (ratio of permeation to reaction of 1-10. For currently available hydrogen separation membrane materials, such values suggest a membrane thickness less than 100 μm of dense ceramic material. The model results also mapped appropriately with experimental data as shown in FIG. 6 which graphically presents experimental results verses chemical model simulated product selectivities as a function of methane conversion, which was varied by changing the reactor residence time at constant temperature (950 K) and inlet methane partial pressure (0:5 bar) and by allowing the number of sites within the reactor to decrease as deactivation occurs.

### EXAMPLE 2

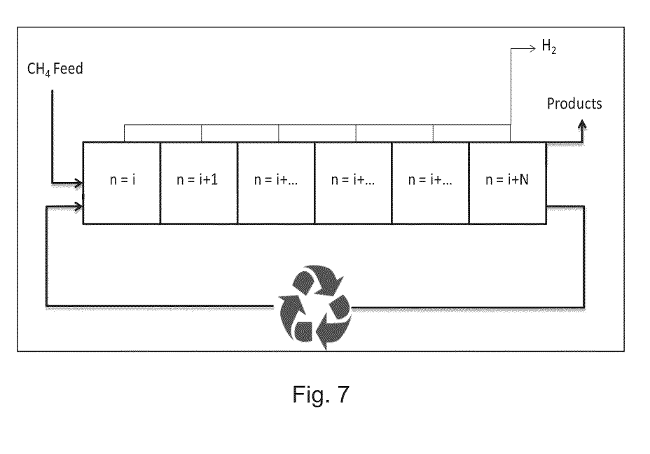

Chemical process simulation software, Aspen® Plus, provided by Aspen Technology, Inc. was used to evaluate methane conversion as a function of percentage hydrogen removal. A dehydroaromatization reactor having a hydrogen separation membrane was simulated using Aspen® Plus in order to approximate the yield of benzene production and methane conversion. Six equilibrium reactors were used in series segregated by separator blocks that were used to approximate the removal of methane. Each reactor was coupled with a separator to simulate a “node” in the membrane reactor, thereby discretizing the reactor. A recycle loop stream was also included in the system allowing for unwanted products to be suppressed in the reactor. A conceptual model of the simulation can be found in FIG. 7.

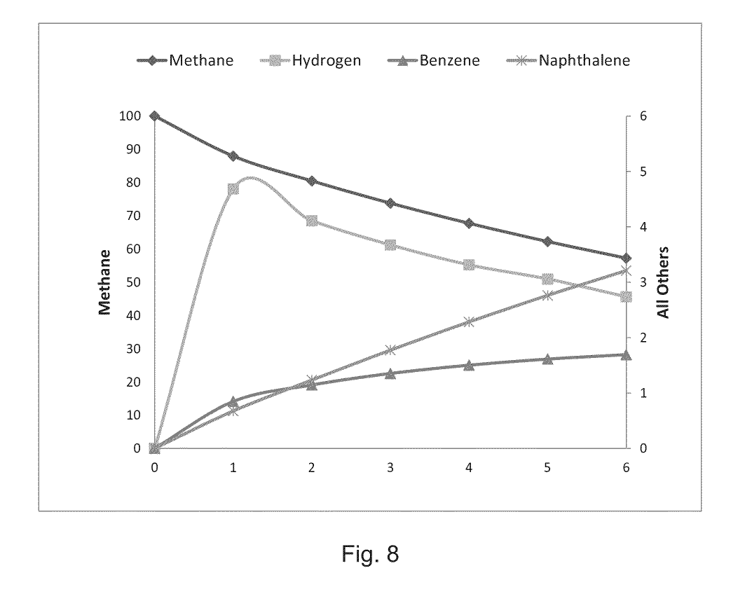

Two different reactions were modeled in this simulation (the dehydration of methane to form benzene and for the formation of naphthalene). The previous stoichiometric equations do not take into account the intermediate products that would be involved with these reactions. This is justified, because it was assumed that each node comes to thermodynamic equilibrium as calculated by Gibb's minimization, wherein only the products and reactants are of concern. By assuming that each node comes to thermodynamic equilibrium, it is also implicitly assumed that the reactions are either infinitely fast or the node is of infinite volume, as well as perfect mixing in the reactor. FIG. 8 shows the results of simulations. It can be observed that 60% methane conversion is possible with removal of a fraction of hydrogen.

While specific embodiments and examples of the present invention have been illustrated and described, numerous modifications come to mind without significantly departing from the spirit of the invention, and the scope of protection is only limited by the scope of the accompanying claims.

1. An apparatus to produce an aromatic hydrocarbon via catalyzed nonoxidative dehydroaromatization (DHA) comprising:
a reaction zone comprising a dehydroaromatization catalyst;
a reactant feed stream inlet that supplies a reactant composition to the reaction zone;
a heater to maintain the reaction zone at a suitable dehydroaromatization temperature;
a product stream exit that removes an aromatic hydrocarbon produced by the nonoxidative dehydroaromatization of the reactant composition from the reaction zone;
a hydrogen separation membrane disposed between the reaction zone and a hydrogen stream exit, wherein the hydrogen separation membrane selectively removes hydrogen produced in the reaction zone; and
a hydrogen recycle stream that diverts a portion of hydrogen from the hydrogen stream exit and adds the portion of hydrogen to the reactant composition supplied to the reaction zone.

2. The apparatus to produce an aromatic hydrocarbon according to claim 1, wherein the dehydroaromatization catalyst comprises a rhenium exchanged zeolite (Re/ZSM-5) catalyst.

3. The apparatus to produce an aromatic hydrocarbon according to claim 1, wherein the hydrogen separation membrane comprises a ceramic membrane that selectively transports H⁺ ions at dehydroaromatization operating temperatures.

4. The apparatus to produce an aromatic hydrocarbon according to claim 1, wherein the hydrogen separation membrane is thermally stable and effective at a temperature above 800° C.

5. The apparatus to produce an aromatic hydrocarbon according to claim 1, wherein the hydrogen separation membrane selectively transports H⁺ ions under a hydrogen partial pressure gradient, a concentration gradient, or an applied voltage.

6. The apparatus to produce an aromatic hydrocarbon according to claim 1, wherein the hydrogen separation membrane comprises a perovskite, a doped cerate, a doped zirconate, or an acidic phosphate.

7. The apparatus to produce an aromatic hydrocarbon according to claim 1, wherein the hydrogen separation membrane comprises a Ba-cerate ceramic composite

8. The apparatus to produce an aromatic hydrocarbon according to claim 1, wherein the reactant comprises C₁-C₄ alkanes.

9. The apparatus to produce an aromatic hydrocarbon according to claim 1, wherein the reactant comprises natural gas.

10. The apparatus to produce an aromatic hydrocarbon according to claim 1, wherein the reactant comprises methane.

11. The apparatus to produce an aromatic hydrocarbon according to claim 1, wherein the dehydroaromatization temperature is in the range from about 500° C. to 1000° C.

12. The apparatus to produce an aromatic hydrocarbon according to claim 1, wherein the dehydroaromatization temperature is in the range from about 700° C. to 900° C.

13. The apparatus to produce an aromatic hydrocarbon according to claim 1, wherein the aromatic hydrocarbon is selected from the group of compounds consisting of benzene, toluene, ethylbenzene, styrene, xylene and naphthalene.

14. A process for catalyzed nonoxidative dehydroaromatization (DHA) of a reactant feed stream comprising C₁-C₄ alkanes to produce a product stream comprising an aromatic hydrocarbon, wherein the process comprises:
contacting the reactant feed stream with a dehydroaromatization catalyst in a reaction zone under conditions to produce the product stream and hydrogen;
continuously removing hydrogen from the reaction zone through a hydrogen separation membrane and collecting the separated hydrogen;
continuously removing the product stream from the reaction zone to recover the aromatic hydrocarbon; and
adding a portion of the separated hydrogen to the reactant feed stream.

15. The process for catalyzed nonoxidative dehydroaromatization according to claim 14, further comprising providing a heater to maintain the reaction zone at a suitable dehydroaromatization temperature.

16. The process for catalyzed nonoxidative dehydroaromatization according to claim 14, further comprising periodically regenerating the dehydroaromatization catalyst by contacting the dehydroaromatization catalyst with hydrogen.

17. The process for catalyzed nonoxidative dehydroaromatization according to claim 14, wherein the dehydroaromatization catalyst comprises a rhenium exchanged zeolite (Re/ZSM-5) catalyst.

18. The process for catalyzed nonoxidative dehydroaromatization according to claim 14, wherein the hydrogen separation membrane comprises a ceramic membrane that selectively transports H⁺ ions at dehydroaromatization operating temperatures.

19. The process for catalyzed nonoxidative dehydroaromatization according to claim 14, wherein the hydrogen separation membrane is thermally stable and effective at a temperature above 800° C.

20. The process for catalyzed nonoxidative dehydroaromatization according to claim 14, wherein the hydrogen separation membrane selectively transports H⁺ ions under a hydrogen partial pressure gradient, a concentration gradient, or an applied voltage.

21. The process for catalyzed nonoxidative dehydroaromatization according to claim 14, wherein the hydrogen separation membrane comprises a perovskite, a doped cerate, a doped zirconate, or an acidic phosphate.

22. The process for catalyzed nonoxidative dehydroaromatization according to claim 14, wherein the hydrogen separation membrane comprises a Ba-cerate ceramic composite.

23. The process for catalyzed nonoxidative dehydroaromatization according to claim 14, wherein the reactant comprises natural gas.

24. The process for catalyzed nonoxidative dehydroaromatization according to claim 14, wherein the reactant comprises methane.

25. The process for catalyzed nonoxidative dehydroaromatization according to claim 14, wherein the dehydroaromatization temperature is in the range from about 500° C. to 1000° C.

26. The process for catalyzed nonoxidative dehydroaromatization according to claim 14, wherein the dehydroaromatization temperature is in the range from about 700° C. to 900° C.

27. The process for catalyzed nonoxidative dehydroaromatization according to claim 14, wherein the aromatic hydrocarbon is selected from the group of compounds consisting of benzene, toluene, ethylbenzene, styrene, xylene and naphthalene.
